(19) United States
(12) Patent Application Publication
(10) Pub. No.: US 2004/0163163 A1
(43) Pub. Date: Aug. 26, 2004

(54) Structural improvement of frameless swimming mask

(75) Inventor: Chih-Cheng Shiue, Rancho Santa Fe, CA (US)

Correspondence Address: BRUCE H. TROXELL, 5205 LEESBURG PIKE, SUITE 1404, FALLS CHURCH, VA, 22041, US

(73) Assignee: QDS Injection Molding LLC.

(21) Appl. No.: 10/369,641

(22) Filed: Feb. 21, 2003

(51) Int. Cl. A61F 9/02

(52) U.S. Cl. 2/428

(57) ABSTRACT

A swimming mask having a plate located between the two lenses. The plate has at least one pair of protuberance that attaches to caverns or holes on the two lenses; the protuberances and caverns or holes combination allows to strengthen the structure of the two lenses and the soft skirt. This creates a useful double lenses frameless diving mask.

### FIELD OF THE INVENTION

The present invention relates to a diving mask, especially, to providing a plate that is between the two lenses. The main purpose is to strengthen the structure of the skirt and lenses (i.e. tempered glass, PC or other transparent lenses) by injection molding.

### BACKGROUND OF THE INVENTION

The usual diving mask structure includes a hard lens, a hard main frame, a hard sub-frame, and a soft skirt. On the other hand, there is also a strap and buckles to tighten the diving mask on the user's head. Mainly the idea is to use the hard main frame and sub-frame to press the soft skirt and the lens strongly in order to prevent leaking water. The odds of this kind of diving mask are large volume and costly when assembling.

Also, there is a kind of frameless (or light-frame) diving mask structure. During the assembly process, the soft skirt and the hard lens are assembled together by using a clip frame or a pair of buckles assembled closed to the two sides of the lens. This way it provides a lighter weight and more compact diving mask. Since it is closer to the user's face and eyes, the vision is much larger.

From the description above, there are also similar structures that do not use clip frame, and assembles the soft skirt and the hard lens together.

Above said “frameless” means a structure that does not have hard mainframe and hard sub-frame at the same time.

Said frameless swimming mask, the specialty of it is that during the process of assembling, special glue is used in the skirt's material. This allows the soft skirt and the lens to join together while injection molding is done at the same time, so that the soft skirt will strongly attach with the lens.

Due to the structural strength problem of the above frameless swimming mask, it can only be a single lens structure. If the structure is built to have double lenses, the structure could be unstable. Especially the soft part between the two lenses can easily fall apart due to external forces, allowing water to easily leak in.

Therefore, the inventor made a significant improvement to these frameless structures. The goal is to invent a frameless swimming mask that has double lenses and stable structure.

### SUMMARY OF THE INVENTION

The present invention is a structure of frameless diving mask, which having a plate or the like between the two lenses. This way the whole structure can be strengthened and the chance of corruption will be decreased. The plate can sustain pressure from external forces, so that the soft skirt can be protected and the hard lens can be stably assembled.

The present invention can join together with the soft skirt when doing injection molding at the same time, when the plate and the two lenses are assembled.

The plate can also be assembled between the two lenses after the two lenses and the soft skirt are assembled.

Plate can also be assembled on a swimming mask with a clip frame, the clip frame can be made of plastic or metal.

By concluding the above descriptions, this present invention provides a two lenses swimming mask that is stronger structurally. Also the two lenses would not be distorted in shape due to external forces. So that this frameless double lenses structure can be used.

Those having skill in the art to which the present invention pertains, will now, as a result of the applicant's teaching herein, perceive various modifications and additions which may be made to the invention.

### BRIEF DESCRIPTION OF THE DRAWINGS

FIG. 1 is a perspective exploded view of the present invention.

FIG. 2 is an assembled view between the plate and the lenses.

FIG. 3 is the perspective exploded view of another embodiment for the diving mask of the present invention.

### DETAILED DESCRIPTION OF THE INVENTION

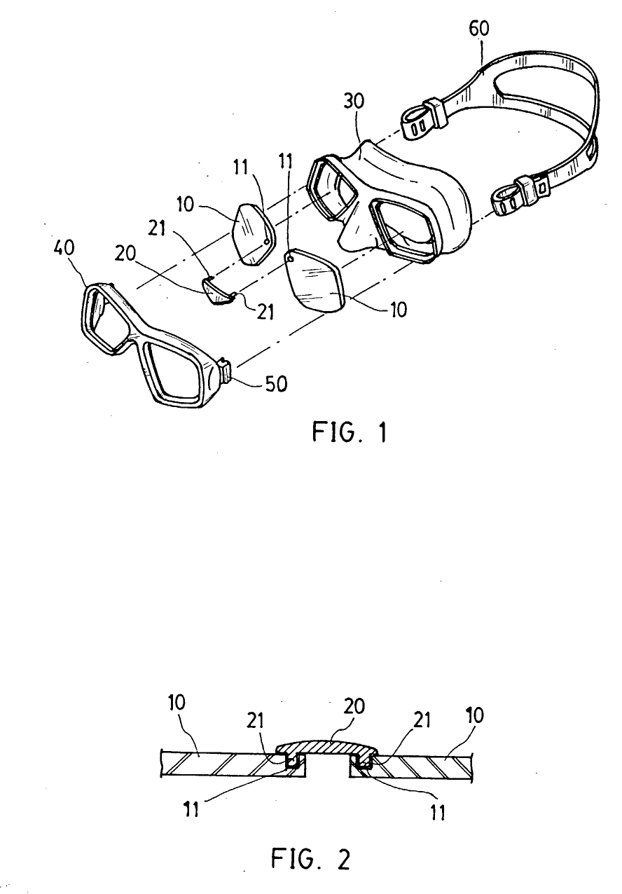

As shown in FIG. 1, the structure of the present invention includes a pair of lenses 10, a plate 20, a soft skirt 30, a clip frame 40, a pair of buckles 50, and a strap 60.

Said pair of lenses 10 has a hard and transparent texture, this allows the user to see behind the mask. The lenses 10 can be flat boarded, a combination of multiple flat boards or even with curvatures. There are caverns 11 at the inner side of both the lenses 10.

Said plate 20 has texture that is sort of hard. There are protuberances 21 on both sides of the plate 20. The positions of protuberance 21 are correspond to the positions of caverns 11, so that the protuberances 21 can be insert to the caverns 11.

Said skirt 30 has soft silicon texture. It can be attached to the edge of lenses 10, this way it prevents water from leaking in.

Said clip frame 40 is used to clip the skirt and the edge of lenses 10. Not only the clip frame 40 can strengthen the structure, it can also protect the edge of the lenses 10. Buckles 50 at assembled on the two sides of the lenses, and these buckles 50 are used to assemble the strap 60 on them.

Said strap 60 is used to tie on users' heads.

The special part of this invention is that the plate 20 positioned between the two lenses 10. The purpose is to stabilize the structure of the mask, especially when the plate 20 is assembled, both the lenses 10 and the skirt 30 can be well attached to each other and prevent water from leaking in.

Referring to FIG. 2, the protuberances 21 on the plate 20 are inserted into the caverns 11 on the lenses 10. This is one of the multiple combinations. Also, the caverns 11 can be holes, so when the protuberances 21 goes through, the mask can be waterproof.

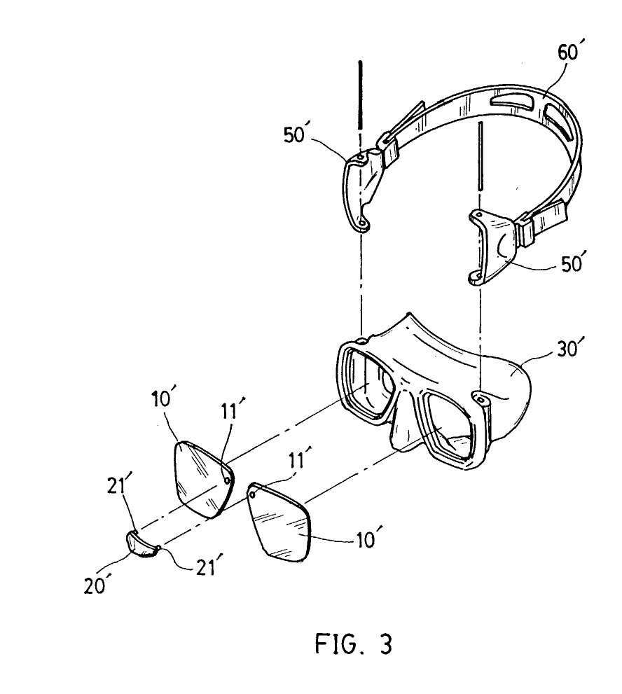

Referring to FIG. 3 is another assembly of the present invention. The structure includes a pair of lenses 10′, a plate 20′, a soft skirt 30′, a pair of buckles 50′, and a strap 60′. The caverns 11′ are on the lenses 10′, and the protuberances 21′ are assembled on the plate 20′. The caverns 11′ and the protuberances 21′ correspond to each other, so that the lenses 10′ and the plate 20′ can attach to each other. The soft skirt is covering and attaching the edge of the lenses 10′. Buckles 50′ and the strap 60′ are located at the skirt 30′, which is at the outer part of both lenses 10′.

As mentioned above, the specialty of this invention is to have a plate assembled between the two lenses. The plate can sustain most of the pressure coming from the external forces. According to the drawings, as long as the specialty of this invention is used in other diving masks, the scope of this invention should cover them all.

What is claimed is:

1. A structural improvement of frameless swimming mask comprising:
a pair of lenses, providing caverns located at the inner sides therein;
a plate between said pair of lenses, both side of said plate having at least one protuberance;
a soft skirt, at least flange to the edge of the lenses;
a pair of buckles with strap which coupling to said mask;
a strap;
Characterized that said plate attaches to the lenses by coupling protuberances and caverns; whereby the structure would be more stronger, and the lenses and the mask can prevent leakage of water.

2. The structural improvement of frameless swimming mask of claim 1, further coupling a clip frame which clip the skirt and lenses, whereby cover said plate and lenses.

3. The structural improvement of frameless swimming mask of claim 1, said caverns can be holes to allow said protuberance insert through.
